(19) United States
(12) Patent Application Publication
(10) Pub. No.: US 2006/0229338 A1
(43) Pub. Date: Oct. 12, 2006

(54) 2,3'-bipyridines derivatives as selective cox-2 inhibitors

(76) Inventors: Juan Francisco Caturla Javaloyes, Barcelona (ES); Graham Warrellow, Barcelona (ES)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/544,360

(22) PCT Filed: Feb. 12, 2004
(86) PCT No.: PCT/EP04/01297

§ 371 Date: Apr. 10, 2006

(30) Foreign Application Priority Data
Feb. 13, 2003 (ES) ES2003300354

(51) Int. Cl. A61K 31/444; C07D 401/04

(52) U.S. Cl. 514/332; 546/257

(57) ABSTRACT

The present invention relates to 2,3′-bipyridines of formula (I), processes for their preparation, pharmaceutical compositions containing them, and their medical uses.

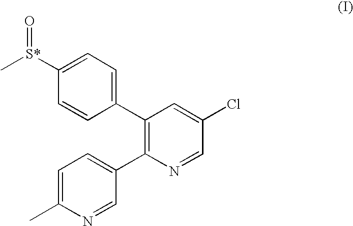

This invention relates to new therapeutically useful 2,3′-bipyridines, to their use as medicaments, to a processes for their preparation and to pharmaceutical compositions containing them.

It is known that non-selective inhibition of the enzyme cyclooxygenase (COX) prevents the overproduction of prostaglandins associated with inflammation, which are mediated by cyclooxygenase-2 (COX-2) but, at the same time, deprives tissues of basal levels of prostaglandins necessary for the health of certain tissues mediated largely by cyclooxygenase-1 (COX-1). Non steroidal anti-inflammatory drugs are non-selective inhibitors of COX and for that reason, have side effects of decreased renal blood flow, decreased platelet function, dyspepsia and gastric ulceration.

We have now found that certain 2,3′-bipyridines selectively inhibit COX-2 in preference to COX-1 and are useful in the treatment of COX-2 mediated diseases, such as inflammation, pain, fever, and asthma with fewer side effects.
Accordingly, the present invention provides compounds of formula (I) 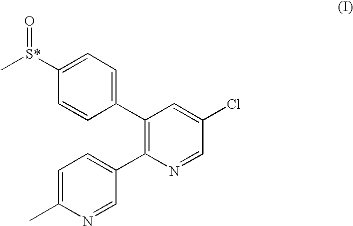
or the salts and N-oxides thereof in the form of any of the two enantiomers or any mixture thereof for use as a medicament.

The compounds of formula (I) have a chiral center in the sulfur atom of the sulfoxide group, shown by an asterisc (*) in the formula, and consequently exist in the form of the two different enantiomers. The two enantiomers and any mixtures thereof are encompassed in the present invention.

Other aspects of the present invention are: a) a process for the preparation of the compounds of formula (I) or the pharmaceutically acceptable salts and N-oxides thereof, b) pharmaceutical compositions comprising an effective amount of said compounds, c) the use of said compounds in the manufacture of a medicament for the treatment of diseases susceptible of being improved by inhibition of the enzyme cyclooxygenase-2 (COX-2); and d) methods of treatment of diseases susceptible to amelioration by inhibition of the enzyme cyclooxygenase-2 (COX-2), which methods comprise the administration of the compounds of the invention to a subject in need of treatment.

It is a preferred embodiment of the present invention that the compounds of formula (I) are in the form of the free base. Alternatively at least one of the two nitrogen atoms of the pyridine ring can be protonised, quaternised or oxidated to yield the corresponding salts or N-oxides.

Particular individual compounds of the invention include:
(R) 5-chloro-6′methyl-3-[4-(methylsulfinyl)phenyl]-2,3′-bipyridine
(S) 5-chloro-6′methyl-3-[4-(methylsulfinyl)phenyl]-2,3′-bipyridine

In another aspect the present invention encompasses a synthetic process for the preparation of the compounds of formula (I) which is depicted in Scheme 1

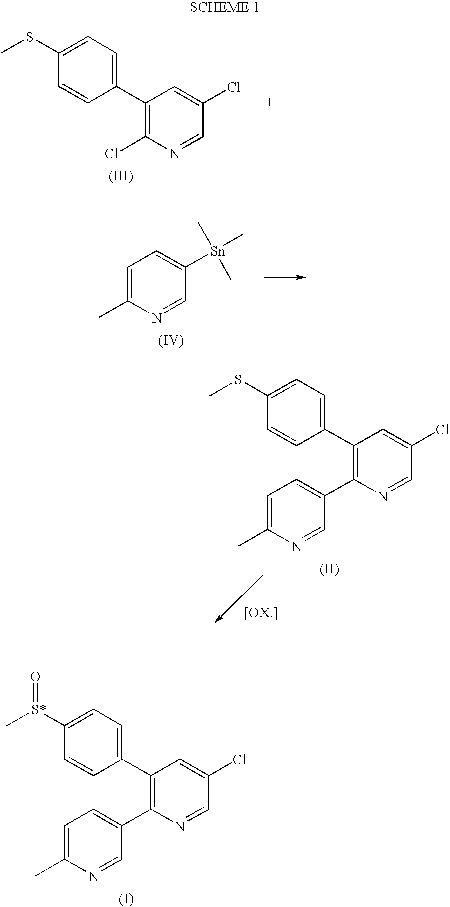

The process shown in Scheme 1 involves the reaction at high temperature such as 100° C. of 2,5-dichloro-3-[4-(methylthio)phenyl]pyridine (III) with (2-Methylpyridin-5-yl)trimethyltin (IV) in the presence of palladium tetrakis(triphenylphosphine) in a solvent to yield 5-chloro-6′methyl-3-[4-(methylthio)phenyl]-2,3′-bipyridine which is isolated and then oxidated to 5-chloro-6′methyl-3-[4-(methylsulfinyl)phenyl]-2,3′-bipyridine (I). The oxidation step can be made under non-stereo specific conditions or under stereo specific conditions.

In the first case the mercapto compound of the previous step is dissolved in methanol an a solution of sodium metaperiodate is added dropwise at a temperature comprised between −15° C. and 10° C., preferably at 0° C. and this mixture is stirred at this temperature for 4 hours at r.t. Then, the reaction is poured into water, extracted with ethyl acetate, the organic solution washed with brine, dried (Na₂SO₄), and the solvent removed under reduced pressure. The residue chromatographically purified yields 5-chloro-6′methyl-3-[4-(methylsulfinyl)phenyl]-2,3′-bipyridine as an off-white solid.

In the second case t-butyl hydroperoxide in nonane and the mercapto compound of the previous step are added successively to a stirred solution of titanium tetraisopropoxide and an optically active diethyl tartrate (either the (R,R) or the (S,S) enantiomers) in dry 1,2-dichloroethane cooled to a temperrature comprised between −30° C. and 30° C., preferably at −20° C. The mixture is stirred at this temperature for 6 h, then washed with an aqueous solution of sodium sulfite and brine. The organic layer is dried (Na₂SO₄) and the solvent removed under reduced pressure. The residue after purification by flash chromatography yields an optically pure enantiomer of 5-chloro-6′methyl-3-[4-(methylsulfinyl)phenyl]-2,3′-bipyridine obtained as an off-white solid.

2,5-dichloro-3-[4-(methylthio)phenyl]pyridine (III) used as a starting product in the process of scheme 1 may be prepared from 3-bromo-5-chloropyridin-2-ol (IV) in a multi-step process depicted in Scheme 2.

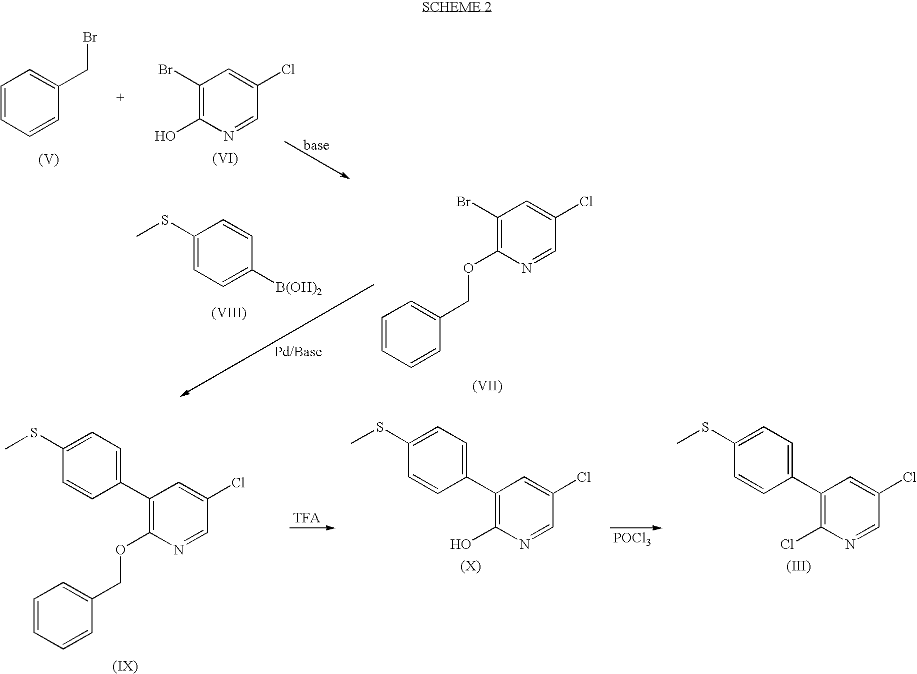

In a first step, treatment of 3-bromo-5-chloropyridin-2-ol (VI) with benzyl bromide (V) in the presence of a base such as silver carbonate yields the benzyl ether (VII) which can be converted to 2-(benzyloxy)-5-chloro-3-[4-(methylthio)phenyl]pyridine (IX) through a palladium-catalyzed coupling with 4-(methylthio)phenyl boronic acid (VIII) in the presence of a suitable base, such as sodium carbonate. The benzyl protecting group can be removed by treatment with an acid such as trifluoroacetic acid to afford 5-chloro-3-[4-(methylthio)phenyl]pyridin-2-ol (X). Heating 5-chloro-3-[4-(methylthio)phenyl]pyridin-2-ol (X) with POCl₃ provides 2,5-dichloro-3-[4-(methylthio)phenyl]pyridine (III).

As used herein, the term pharmaceutically acceptable salt embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

Other salts according to the invention are quaternary ammonium in which at least one of the nitrogen atoms is quaternised with a C₁-C₆ alkyl group. Such compounds may be obtained by reacting the free base compounds of the present invention with quaternising agents, preferably with C₁-C₆ alkyl halides under conventional quaternising conditions.

In the quaternary ammonium compounds of the present invention, an equivalent of an anion (X⁻) is associated with the positive charge of the N atom. X⁻ may be an anion of various mineral acids such as, for example, chloride, bromide, iodide, sulphate, nitrate, phosphate, or an anion of an organic acid such as, for example, acetate, trifluoroacetate, maleate, fumarate, citrate, oxalate, succinate, tartrate, malate, mandelate, methanesulfonate and p-toluenesulfonate. X⁻ is preferably an anion selected from chloride, bromide, iodide, sulphate, nitrate, acetate, trifluoroacetate, methanesulfonate, maleate, oxalate or succinate. More preferably X⁻ is chloride, bromide, trifluoroacetate or methanesulfonate.

The N-oxide compounds of the present invention may be formed from the free base compounds using a convenient oxidising agent.

Pharmacological Activity

The following biological tests and data further illustrate this invention.

### COX-1 and COX-2 Activities in Human Whole Blood

Fresh blood from healthy volunteers who had not taken any non-steroidal anti-inflammatory drugs for at least 7 days prior to blood extraction was collected in heparinized tubes (20 units of heparin per ml). For the COX-1 activity determination, 500 μl aliquots of blood were incubated with either 5 μl vehicle (dimethylsulphoxide) or 5 μl of a test compound for 24 h at 37° C. Calcium ionophore A23187 (25 μM) was added 20 min before stopping the incubation. Plasma was separated by centrifugation (10 min at 13000 rpm) and kept at −30° C. until TXB₂ levels were measured using an enzyme immunoassay kit (EIA).

The effect of the compounds was evaluated by incubating each compound at five to six different concentrations with triplicate determinations. IC₅₀ values were obtained by non-linear regression using InPlot, GraphPad software on an IBM computer.

For the COX-2 activity determination, 500 μl aliquots of blood were incubated in the presence of LPS (10 μg/ml) for 24 h at 37° C. in order to induce the COX-2 expression (Patriagnani et al., J. Pharm. Exper. Ther. 271; 1705-1712 (1994)). Plasma was separated by centrifugation (10 min at 13000 rpm) and kept at −30° C. until PGE₂ levels were measured using an enzyme immunoassay kit (EIA). The effects of inhibitors were studied by incubating each compound (5 μl aliquots) at five to six different concentrations with triplicate determinations in the presence of LPS for 24 hours. IC₅₀ values were obtained by non-linear regression using InPlot, GraphPad software on an IBM computer.

The results obtained from the biological assays are shown in Table 1 which shows the inhibition of COX-1 and COX-2 obtained with the racemic mixture of 5-chloro-6′-methyl-3-[4-(methylsulfinyl)phenyl]-2,3′-bipyridine.

TABLE I

| | COX-1 | COX-2 | Ratio |
|---|---|---|---|
| Example | IC₅₀ μM | IC₅₀ μM | COX-1/COX-2 |
| 5-chloro-6′methyl-3-[4- | 45.5 | 3.7 | 12.3 |
| (methylsulfinyl)phenyl]-2,3′- | | | |
| bipyridine (racemate) | | | |
| 5-chloro-6′methyl-3-[4- | 20.2 | — | — |
| (methylsulfinyl)phenyl]-2,3′- | | | |
| bipyridine (Enantiomer 1a) | | | |
| 5-chloro-6′methyl-3-[4- | 77.8 | 3.5 | 22.2 |
| (methylsulfinyl)phenyl]-2,3′- | | | |
| bipyridine (Enantiomer 1b) | | | |

As shown in Table 1, the 2,3′-bipyridines (I) are potent and selective COX-2 inhibitors. Thus the compounds of the invention are preferably selective inhibitors of mammalian COX-2, for example human COX-2.

The compounds of the invention also preferably have low inhibitory activity toward mammalian COX-1, for example human COX-1. Inhibitory activity can typically be measured by in vitro assays, for example as described above. Some of the compounds of the present invention have also shown an interesting pharmacokinetic profile.

Preferred compounds of the invention have an IC50 value for COX-2 of less than 50 μM, preferably less than 10 μM more preferably less than 5 μM. Preferred compounds of the invention also have an IC₅₀ value for COX-1 of greater than 10 μM, preferably greater than 20 μM. As an indicator of selectivity for inhibition of COX-2 over COX-1, the ratio of COX-1/COX-2 IC₅₀ values is preferably greater than 10 more preferably greater than 20 still more preferably greater than 30.

The present invention further provides a compound of formula (I) for use in a method of treatment of the human or animal body by therapy, in particular for the treatment of pain, fever or inflammation, to inhibit prostanoid-induced smooth muscle contraction or for the prevention or treatment of colorectal cancer or neurodegenerative diseases, for example, Alzheimer disease.

The present invention further provides the use of a compound of formula (I) in the manufacture of a medicament for the treatment of pain, fever or inflammation, to inhibit prostanoid-induced smooth muscle contraction or for the prevention or treatment of colorectal cancer.

The compounds of formula (I) are useful for relief of pain, fever and inflammation of a variety of conditions including rheumatic fever, symptoms associated with influenza or other viral infections, common cold, low back and neck pain, dysmenorrhoea, headache, toothache, sprains and strains, myositis, neuralgia, synovitis, bursitis, tendinitis, injuries, following surgical and dental procedures and arthritis including rheumatoid arthritis, osteoarthritis, gouty arthritis, spondyloarthopathies, systemic lupus erythematosus and juvenile arthritis. They may also be used in the treatment of skin inflammation disorders such as psoriasis, eczema, burning and dermatitis. In addition, such compounds may be used for the prevention or treatment of colorectal cancer or neurodegenerative diseases, for example, Alzheimer disease.

The compounds of formula (I) will also inhibit prostanoid-induced smooth muscle contraction and therefore may be used in the treatment of dysmenorrhoea, premature labour, asthma and bronchitis.

The compounds of formula (I) can be used as alternative to conventional non-steroidal anti-inflammatory drugs, particularly where such non-steroidal anti-inflammatory drugs may be contraindicated such as the treatment of patients with gastrointestinal disorders including peptic ulcers, gastritis, regional enteritis, ulcerative colitis, diverticulitis, Crohn's disease, inflammatory bowel syndrome and irritable bowl syndrome, gastrointestinal bleeding and coagulation disorders, kidney disease (e.g. impaired renal function), those prior to surgery or taking anticoagulants, and those susceptible to non-steroidal anti-inflammatory drugs induced. asthma.

The compounds can further be used to treat inflammation in diseases such as vascular diseases, migraine headaches, periarteritis nodosa, thyroiditis, aplastic anaemia, Hodgkin's disease, scleroderma, type I diabetes, myasthenia gravis, sarcoidosis, nephrotic syndrome, Behcet's syndrome, polymyositis, hypersensitivity, conjunctivitis, gingivitis and myocardial ischaemia.

Compounds of the present invention are inhibitors of cyclooxygenase-2 enzyme and are thereby useful to treat the cyclooxygenase-2 mediated diseases enumerated above.

Accordingly, the compounds of the present invention and pharmaceutical compositions comprising such compounds may be used in a method of treatment of disorders of the human body which comprises administering to a patient requiring such treatment an effective amount of such compounds.

The present invention also provides pharmaceutical compositions, which comprise, as an active ingredient, at least a compound of formula (I) or a pharmacologically acceptable salt or N-oxide thereof in association with a pharmaceutically acceptable excipient such as a carrier or diluent. The active ingredient may comprise 0.001% to 99% by weight, preferably 0.01% to 90% by weight of the composition depending upon the nature of the formulation and whether further dilution is to be made prior to application.

Preferably the compositions are made up in a form suitable for oral, topical, nasal, inhalation, rectal, percutaneous or injectable administration.

The pharmaceutically acceptable excipients that are admixed with the active compound or salts of such compound, to form the compositions of this invention are well known per se and the actual excipients used depend inter alia on the intended method of administering the compositions.

Compositions of this invention are preferably adapted for injectable and per os administration. In this case, the compositions for oral administration may take the form of tablets, retard tablets, sublingual tablets, capsules or liquid preparations, such as mixtures, elixirs, syrups or suspensions, all containing the compound of the invention; such preparations may be made by methods well-known in the art.

The diluents that may be used in the preparation of the compositions include those liquid and solid diluents that are compatible with the active ingredient, together with colouring or flavouring agents, if desired. Tablets or capsules may conveniently contain between 2 and 500 mg of active ingredient or the equivalent amount of a salt thereof.

The liquid composition adapted for oral use may be in the form of solutions or suspensions. The solutions may be aqueous solutions of a soluble salt or other derivative of the active compound in association with, for example, sucrose to form a syrup. The suspensions may comprise an insoluble active compound of the invention or a pharmaceutically acceptable salt thereof in association with water, together with a suspending agent or flavouring agent.

Compositions for parenteral injection may be prepared from soluble salts, which may or may not be freeze-dried and which may be dissolved in pyrogen free aqueous media or other appropriate parenteral injection fluid.

Effective doses are normally in the range of 10-600 mg of active ingredient per day. Daily dosage may be administered in one or more treatments, preferably from 1 to 4 treatments, per day.

The invention is illustrated by the following Preparation and Examples, which do not limit the scope of the invention in any way.

¹H Nuclear Magnetic Resonance Spectra were recorded on a Varian Gemini 300 spectrometer. Melting points were recorded using a Perkin Elmer DSC-7 apparatus. Optical rotations were determined on a Perkin Elmer 241MC Polarimeter. Enantiomeric purities were determined by capillary electrophoresis on an Agilent 3D (Agilent Technologies, Waldbronn, Germany), using a diode array detector and a capillary of melted silica (56 cm longitude, 50 microm internal diameter). The conditions used were the following: buffer (phosphoric acid 20 mM adjusted to pH 3.0 with triethanolamine, sulphobutylether cyclodextrin of substitution grade 7 (SBE-7CD), 10% acetonitrile); voltage (30 kV with negative polarity); temperature (20° C.); wavelength (200 nm (15 nm bandwidth) with a reference of 400 nm (80 nm bandwidth)).

### EXAMPLES

### Preparation 1

5-Chloro-6′-methyl-3-[4-(methylthio)phenyl)-2,3′-bipyridine

a) A mixture of 5-chloro-2-hydroxypyridine (20.1 g, 155.4 mmol) and bromine (11.9 ml) in acetic acid (250 m) was stirred at r.t. for 2 hours. Then, the solvent was evaporated in vacuo and ethyl acetate (600 ml) and saturated sodium bicarbonate (300 ml) were added. The organic layer was washed with saturated sodium bicarbonate (2×200 ml), brine and was dried (Na₂SO₄) and concentrated to give a solid that was crystallized from hexane/diethyl ether. The solid 3-bromo-5-chloro-2-hydroxypyridine (21.3 g, 66%) so obtained was used in the subsequent reaction.

b) A mixture of 3-bromo-5-chloro-2-hydroxypyridine (21.28 g, 0.10 mmol), benzyl bromide (13.3 ml) and silver carbonate (25.6 g) in toluene (600 ml) was heated at 70° C. for 1 hour. The mixture was cooled to r.t. and then filtered through a bed of Celite. The filtrate was concentrated and the residual off-white solid was recrystallized from pentane to provide 2-benzyloxy-3-bromo-5-chloropyridine as a white solid (27.12 g, 89%).

c) A mixture of 2-benzyloxy-3-bromo-5-chloropyridine (27.12 g, 90.9 mmol), methyltiobenzene boronic acid (Li, et al. J. Med. Chem. 1995, 38, 4570) (18.3 g), 2M aqueous sodium carbonate (120 ml) and palladium tetrakis(triphenylphosphine) (1.05 g) in ethanol/toluene (240 ml, 1:1) was heated at reflux for 15 h. The mixture was cooled to r.t., filtered through celite and the filtrate was washed with water (2×200 ml) and brine. The organics were dried (Na₂SO₄) and concentrated to give a residue which was crystallized from hexane/diethyl ether affording 2-benzyloxy-5-chloro-3-[4-(methylthio)phenyl]pyridine (27.2 g, 88%) as a beige solid.

d) A solution of 2-benzyloxy-5-chloro-3-[4-(methylthio)phenyl]pyridine (3.0 g, 8.78 mmol) in trifluoroacetic acid (12 ml) was stirred at 40° C. for 15 min and then poured into ice/water. This mixture was extracted with ethyl acetate (100 ml) and the organic layer was washed with saturated sodium bicarbonate (3×50 ml) and brine, dried (Na₂SO₄) and concentrated to give a residue that was crystallized from hexane/diethyl ether. 5-Chloro-2-hydroxy-3-[4-(methylthio)phenyl]pyridine (1.24 g, 56%) was obtained as a solid.

e) The crude 5-chloro-2-hydroxy-3-[4-(methylthio)phenyl]pyridine (1.24 g) from step d) was heated in a sealed bomb at 150° C. with POCl₃ (10 ml) for 15 hours. After cooling to r.t. the excess POCl₃ was removed by distillation under vacuum. The residue was diluted with ethyl acetate and water and then neutralized with saturated sodium bicarbonate to pH˜7. The organics were removed, washed with brine and concentrated. The residual solid was recrystallized from hexane/diethyl ether to provide 2,5-dichloro-3-[4-(methylthio)phenyl]pyridine as a white solid (1.12 g, 84%).

f) To a mixture of 5-hydroxy-2-methylpyridine (20 g, 183 mmol) and pyridine (19 ml) in dichloromethane (1 l) at 0° C. was added trifluoromethanesulfonic acid anhydride (34 ml). The mixture was stirred at this temperature for 45 min and then at r.t. for 45 min. Ammonium acetate (25%) was added and the organics were removed and washed with 1 N HCl and brine, dried (Na₂SO₄) and concentrated. 6-Methylpyridin-3-yl trifluoromethanesulfonate was obtained as a beige liquid (40.5 g, 92%) that was used as such.

g) A mixture of 6-methylpyridin-3-yl trifluoromethanesulfonate (40.5 g, 168 mmol), hexamethylditin (55 g), lithium chloride (21.4 g) and palladium tetrakis(triphenylphosphine) (4.4 g) was heated at reflux for 3 days and then cooled to r.t. A pH=7 phosphate buffer solution was added and this mixture was extracted with ethyl acetate. The organic layer was washed with brine, dried (Na₂SO₄) and concentrated. Flash chromatography eluting with hexane/ethyl acetate (4/1) of the residue provided (2-methylpyridin-5-yl)trimethyltin as a pale yellow oil (9.49 g, 22%).

h) A mixture of 2,5-dichloro-3-[4-(methylthio)phenyl]pyridine from step e) (0.29 g, 1.1 mmol), (2-methylpyridin-5-yl)trimethyltin (0.54 g) and palladium tetrakis(triphenylphosphine) (0.12 g) in N-methylpyrrolidone (6 ml) was heated at 100° C. for 15 hours. The mixture was cooled to r.t., diluted with ethyl acetate and filtered through a bed of Celite. The filtrate was washed with water (70 ml), and then extracted with 1N HCl (70 ml). The aqueous phase was neutralized with 8 N sodium hydroxide and then extracted with ethyl acetate (2×70 mL). The organics were washed with brine, dried (Na₂SO₄) and concentrated and the residue subjected to flash chromatography eluting with hexane/ethyl acetate (1/1), to provide the title compound (0.2 g, 59%) as a white solid.

δ (DMSO): 2.44 (s, 3H), 2.48 (s, 3H), 7.16-7.25 (m, 5H), 7.58 (dd, J=7.8, 2.4 Hz, 1H), 8.00 (d, J=2.4 Hz, 1H), 8.31 (d, J=2.7 Hz, 1H), 8.75 (d, J=2.4 Hz, 1H).

### Example 1

### 5-Chloro-6′-methyl-3-[4-(methylsulfinyl)phenyl]-2,3′-bipyridine

To a solution of the title compound of Preparation 1 (1.0 g, 3.06 mmol) in methanol (14 ml) was added dropwise a solution of sodium metaperiodate (0.65 g) in water (8 ml) at 0° C. and this mixture was stirred for 4 hours at r.t. Then, the reaction was poured into water, extracted with ethyl acetate (3×100 ml), the organic solution washed with brine, dried (Na₂SO₄), and the solvent removed under reduced pressure. The residue was recrystallized from hexane/ethyl acetate/diethyl ether affording 5-chloro-6′-methyl-3-[4-(methylsulfinyl)phenyl]-2,3′-bipyridine (0.73 g, 70%) as an off-white solid. m.p.: 122-123° C.

δ (DMSO): 2.44 (s, 3H), 2.77 (s, 3H), 7.18 (d, J=8.1 Hz, 1H), 7.47 (d, J=8.3 Hz, 2H), 7.54 (dd, J=8.1, 2.4 Hz, 1H), 7.67 (d, J=8.3 Hz, 2H), 8.09 (d, J=2.1 Hz, 1H), 8.32 (d, J=2.1 Hz, 1H), 8.80 (d, J=2.4 Hz, 1H).

### Example 2

### 5-Chloro-6′-methyl-3-[4-(methylsulfinyl)phenyl]-2,3′-bipyridine (Enantiomer 1a)

To a stirred solution of titanium tetraisopropoxide (1.05 ml, 3.5 mmol) and (R,R)-diethyl tartrate (2.45 ml, 14.2 mmol) in dry 1,2-dichloroethane (25 ml) cooled to −20° C. were added successively t-butyl hydroperoxide 5.5 M in nonane (1.29 ml, 7.1 mmol) and the title compound of Preparation 1 (1.14 g, 3.5 mmol). The mixture was stirred at −20° C. for 6 h, then washed with a 5% aqueous solution of sodium sulfite (50 ml) and brine. The organic layer was dried (Na₂SO₄) and the solvent removed under reduced pressure. The residue was purified by flash chromatography and ethyl acetate/methanol (8/2) as eluent. 5-Chloro-6′-methyl-3-[4-(methylsulfinyl)phenyl]-2,3′-bipyridine (0.63 g, 60%, 100% ee) in the form of enantiomer 1a was obtained as an off-white solid.

[α]_{D}²²=+48.7 (c 0.25, MeOH)

m.p.: 122-123° C.

δ (DMSO): 2.44 (s, 3H), 2.77 (s, 3H), 7.18 (d, J=8.1 Hz, 1H), 7.47 (d, J=8.3 Hz, 2H), 7.54 (dd, J=8.1, 2.4 Hz, 1H), 7.67 (d, J=8.3 Hz, 2H), 8.09 (d, J=2.1 Hz, 1H), 8.32 (d, J=2.1 Hz, 1H), 8.80 (d, J=2.4 Hz, 1H).

### Example 3

### 5-Chloro-6′-methyl-3-[4-(methylsulfinyl)phenyl]-2,3′-bipyridine (Enantiomer 1b)

Obtained as an off-white solid (51%, 100% ee) from the title compound of Preparation 1 and (S,S)-diethyl tartrate in the form of enantiomer 1b by the procedure described in Example 2.

[α]_{D}²²=−50.7 (c 0.25, MeOH)

m.p.: 122-123° C.

δ (DMSO): 2.44 (s, 3H), 2.77 (s, 3H), 7.18 (d, J=8.1 Hz, 1H), 7.47 (d, J=8.3 Hz, 2H), 7.54 (dd, J=8.1, 2.4 Hz, 1H), 7.67 (d, J=8.3 Hz, 2H), 8.09 (d, J=2.1 Hz, 1H), 8.32 (d, J=2.1 Hz, 1H), 8.80 (d, J=2.4 Hz, 1H).

### Composition Examples

### Composition Example 1

### Preparation of Tablets

Formulation:

| | | |
|---|---|---|
| Compound of the present invention | 5.0 | mg |
| Lactose | 113.6 | mg |
| Microcrystalline cellulose | 28.4 | mg |
| Light silicic anhydride | 1.5 | mg |
| Magnesium stearate | 1.5 | mg |

Using a mixer machine, 15 g of the compound of the present invention are mixed with 340.8 g of lactose and 85.2 9 of microcrystalline cellulose. The mixture is subjected to compression moulding using a roller compactor to give a flake-like compressed material. The flake-like compressed material is pulverised using a hammer mill, and the pulverised material is screened through a 20 mesh screen. A 4.5 g portion of light silicic anhydride and 4.5 g of magnesium stearate are added to the screened material and mixed. The mixed product is subjected to a tablet making machine equipped with a die/punch system of 7.5 mm in diameter, thereby obtaining 3,000 tablets each having 150 mg in weight.

### Composition Example 2

### Preparation of Coated Tablets

Formulation:

| | |
|---|---|
| Compound of the present invention | 5.0 mg |
| Lactose | 95.2 mg |
| Corn starch | 40.8 mg |
| Polyvinylpyrrolidone K25 | 7.5 mg |
| Magnesium stearate | 1.5 mg |
| Hydroxypropylcellulose | 2.3 mg |
| Polyethylene glycol 6000 | 0.4 mg |
| Titanium dioxide | 1.1 mg |
| Purified talc | 0.7 mg |

Using a fluidised bed granulating machine, 15 g of the compound of the present invention are mixed with 285.6 g of lactose and 122.4 g of corn starch. Separately, 22.5 g of polyvinylpyrrolidone is dissolved in 127.5 g of water to prepare a binding solution. Using a fluidised bed granulating machine, the binding solution is sprayed on the above mixture to give granulates. A 4.5 g portion of magnesium stearate is added to the obtained granulates and mixed. The obtained mixture is subjected to a tablet making machine equipped with a die/punch biconcave system of 6.5 mm in diameter, thereby obtaining 3,000 tablets, each having 150 mg in weight.

Separately, a coating solution is prepared by suspending 6.9 g of hydroxypropylmethyl-cellulose 2910, 1.2 g of polyethylene glycol 6000, 3.3 g of titanium dioxide and 2.1 g of purified talc in 72.6 g of water. Using a High Coated, the 3,000 tablets prepared above are coated with the coating solution to give film-coated tablets, each having 154.5 mg in weight.

### Composition Example 3

### Preparation of Capsules

Formulation:

| | | |
|---|---|---|
| Compound of the present invention | 5.0 | mg |
| Lactose monohydrate | 200 | mg |
| Colloidal silicon dioxide | 2 | mg |
| Corn starch | 20 | mg |
| Magnesium stearate | 4 | mg |

25 g of active compound, 1 Kg of lactose monohydrate, 10 g of colloidal silicon dioxide, 100 g of corn starch and 20 g of magnesium stearate are mixed. The mixture is sieved through a 60 mesh sieve, and then filled into 5,000 gelatine capsules.

### Composition Example 4

### Preparation of a cream

Formulation:

| | |
|---|---|
| Compound of the present invention | 1% |
| Cetyl alcohol | 3% |
| Stearyl alcohol | 4% |
| Gliceryl monostearate | 4% |
| Sorbitan monostearate | 0.8% |
| Sorbitan monostearate POE | 0.8% |
| Liquid vaseline | 5% |
| Methylparaben | 0.18% |
| Propylparaben | 0.02% |
| Glycerine | 15% |
| Purified water csp. | 100% |

An oil-in-water emulsion cream is prepared with the ingredients listed above, using conventional methods.

1. A compound of formula (I): 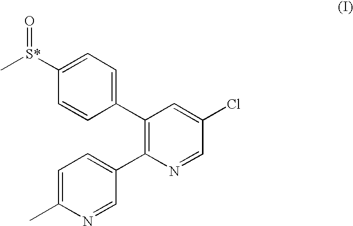
wherein the compound of formula (I) is in the form of any of the two different enantiomers;
or a pharmaceutically acceptable salt thereof; or a N-oxide thereof; or a mixture thereof of any such compounds in any ratio.

2. A compound according to claim 1 which is in the form of the free base.

3. A compound according to claim 1, chosen from:
(R) 5-chloro-6′-methyl-3-[4-(methylsulfinyl)phenyl]-2,3′-bipyridine; and
(S) 5-chloro-6′-methyl-3-[4-(methylsulfinyl)phenyl]-2,3′-bipyridine.

4. (canceled)

5. A process for the preparation of a compound as claimed in claim 1, comprising:
reacting a compound of formula (II) 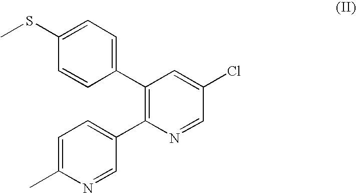
with an oxidizing agent to produce a compound of formula (I), and
optionally converting the compound of formula (I) into a pharmaceutically acceptable salt or into a N-oxide.

6. A process according to claim 5, wherein the oxidizing agent is chosen from:
(a) sodium metaperiodate; and
(b) a mixture of titanium tetraisopropoxide, t-butyl hydroperoxide and either the (R,R) or the (S,S) form of diethyl tartrate.

7. A process according to claim 6, wherein the reaction takes place in at least one chlorinated solvent or in a mixture of at least one chlorinated solvent and at least one C₁-C₄ alcohol.

8. A process according to claim 7, wherein the at least one chlorinated solvent is chosen from 1,2-dichloroethane, methylene chloride, chloroform, and mixtures thereof.

9. A pharmaceutical composition comprising at least one compound according to claim 1 and at least one pharmaceutically acceptable diluent or carrier.

10. A medicament comprising at least one compound according to claim 1.

11. A pharmaceutical composition comprising at least one compound according to claim 3 and at least one pharmaceutically acceptable diluent or carrier.

12. A method for treating a subject afflicted with pathological condition or disease susceptible to amelioration by inhibition of the enzyme cyclooxygenase-2 (COX-2), comprising administering to the subject an effective amount of a compound according to claim 1.

13. A method according to claim 12, wherein the pathological condition or disease is chosen from pain, fever, inflammation, prostanoid-induced smooth muscle contraction, colorectal cancer, and neurodegenerative diseases.

14. A method for treating a subject afflicted with a pathological condition or disease susceptible of amelioration by inhibition of the enzyme cyclooxygenase-2 (COX-2), comprising administering to said subject an effective amount of a composition according to claim 11.

15. A method according to claim 14, wherein the pathological condition or disease is chosen from pain, fever, inflammation, prostanoid-induced smooth muscle contraction, colorectal cancer, and neurodegenerative diseases.

16. A compound according to claim 1, wherein the pharmaceutically acceptable salt is a quaternary ammonium salt.

17. A compound according to claim 16, wherein the quaternary ammonium nitrogen is bound to a C₁-C₆ alkyl group.
